SEQUENCE LISTING (15 sequences)

SEQ ID NO: 1 - DNA, 351 bp, Homo sapiens
atggattactacagaaaatatgcagctatctttctggtcacattgtcggtctttctgcatgttctccattccgctcctgatgtgcaggattgcccagaatgcacgctacaggaaaacccattcttctcccagccgggtgccccaatacttcagtgcatgggctgctgcttctctagagcatatcccactccactaaggtccaagaagacgatgttggtccaaaagaacgtcacctcagagtccacttgctgtgtagctaaatcatataacagggtcacagtaatggggggtttcaaagtggagaaccacacggcgtgccactgcagtacttgttattatcacaaatcttaa

SEQ ID NO: 2 - DNA, 390 bp, Homo sapiens
atgaagacactccagtttttcttccttttctgttgctggaaagcaatctgctgcaatagctgtgagctgaccaacatcaccattgcaatagagaaagaagaatgtcgtttctgcataagcatcaacaccacttggtgtgctggctactgctacaccagggatctggtgtataaggacccagccaggcccaaaatccagaaaacatgtaccttcaaggaactggtatatgaaacagtgagagtgcccggctgtgctcaccatgcagattccttgtatacatacccagtggccacccaggtgcactgtggcaagtgtgacagcgacagcactgattgtactgtgcgaggcctggggcccagctactgctcctttggtgaaatgaaagaataa

SEQ ID NO: 3 - DNA, 27 bp, Artificial Sequence
  sense primer for human FSH alpha subunit
agcgccatggattactacagaaaatat

SEQ ID NO: 4 - DNA, 37 bp, Artificial Sequence
  antisense primer for human FSH alpha subunit
gggggatccgggcccttaagatttgtgataattaaca

SEQ ID NO: 5 - DNA, 33 bp, Artificial Sequence
  sense primer for human FSH beta subunit
gggggcggccgcaggatgaagacactccagttt

SEQ ID NO: 6 - DNA, 27 bp, Artificial Sequence
  antisense primer for human FSH beta subunit
ggggatatcttattctttcatttcacc

SEQ ID NO: 7 - DNA, 592 bp, Encephalomyocarditis virus
gatatcgaattccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataaatggcc

SEQ ID NO: 8 - DNA, 654 bp, Cytomegalovirus
  promoter (1)..(654)
gatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactgcttatcgaaatt

SEQ ID NO: 9 - DNA, 441 bp, Artificial Sequence
  Adenovirus tripartite leader sequence
tcgatactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgca

SEQ ID NO: 10 - DNA, 19 bp, Artificial Sequence
  sense primer for tripartite leader sequence
gatatcgatactctcttcc

SEQ ID NO: 11 - DNA, 27 bp, Artificial Sequence
  antisense primer for tripartite leader sequence
gcgtcgacctgcagttggacctgggag

SEQ ID NO: 12 - DNA, 564 bp, Artificial Sequence
  dihydrofolate reductase
atggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaa

SEQ ID NO: 13 - DNA, 130 bp, Simian virus 40
  polyA_signal (1)..(130)
aacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 14 - DNA, 232 bp, Artificial Sequence
  bovine growth hormone
ctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 15 - DNA, 10 bp, Artificial Sequence
  BamH I linker
cgggatcccg

(19) United States
(12) Patent Application Publication
(10) Pub. No.: US 2006/0223144 A1
(43) Pub. Date: Oct. 5, 2006

(54) Method for mass production of human pollicle stimulating hormone

(75) Inventors: Se Hwan Yang, Gyeongsangbuk-do (KR); Young Chul Sung, Gyeongsangbuk-do (KR); Kyu-Heum Na, Kyunggi-do (KR); Sung Hee Lee, Gwanak-ku (KR); Won Bae Kim, Seoul (KR)

Correspondence Address: JHK LAW, P.O. BOX 1078, LA CANADA, CA, 91012-1078, US

(73) Assignee: Progen Co., Ltd., Kyunggi-do (KR)

(21) Appl. No.: 10/595,200

(22) PCT Filed: Sep. 24, 2004
(86) PCT No.: PCT/KR04/02474

§ 371 Date: Mar. 22, 2006

(30) Foreign Application Priority Data
Oct. 2, 2003 (KR) 10-2003-0068641

(51) Int. Cl. C12P 21/06; C07H 21/04; C12N 15/09; C12N 5/06; C07K 14/59

(52) U.S. Cl. 435/69.4; 435/320.1; 435/358; 530/399; 536/23.5

(57) ABSTRACT

The present invention relates to a method for mass-production of human follicle stimulating hormone, more precisely, to an expression vector containing human follicle stimulating hormone gene, a transformant transfected with the expression vector and a method for mass-production of human follicle stimulating hormone by using the same. Human follicle stimulating hormone can be produced largely by using an expression vector and a transformant of the present invention. Therefore the expression vector and the transformant of the present invention can be effectively used for the treatment of infertility.

### TECHNICAL FIELD

The present invention relates to a method for mass-production of human follicle stimulating hormone, more precisely, to an expression vector containing human follicle stimulating hormone gene, a transformant transfected with the expression vector and a method for mass-production of human follicle stimulating hormone by using the same.

### BACKGROUND ART

Follicle stimulating hormone (referred as “FSH” hereinafter), having a molecular weight of 32,600 Da, is a glycoprotein secreted in anterior pituitary, more precisely, a heterodimeric glycoprotein in which alpha and beta subunits are combined each other by a non-covalent bond. Heterodimeric glycoproteins are exemplified by FSH, luteinizing hormone (referred as “LH” hereinafter) which is a pituitary glycoprotein, human chorionic gonadotropin (referred as “hCG” hereinafter) which is a placental glycoprotein, thyroid stimulating hormone (referred as “TSH” hereinafter), factor VIII and IL-12, etc. The alpha subunit of FSH is very similar in structure and in immunological aspects to LH, TSH and hCG. But, the beta subunit is different from every hormone, meaning that each hormone has unique biological and immunological characteristics owing to the different properties of beta subunit (Pierce. J. G and Parsons. T. F., Ann. Rev. Biochem., 50:465-495, 1981). In particular, human FSH (hFSH) stimulates follicle of an ovary to make the follicle be growing, leading to the ovulation. Thus, it has been in clinical use for the treatment of sterility. FSH is obtained from urine of pregnant women, but the obtainable amount is very small and the separation is difficult. Therefore, it is urgently required to develop a method for mass-production of the FSH by using a genetic recombinant technique.

In order to prepare a transformed cell line expressing a glycoprotein hormone by taking advantage of a genetic recombinant technique, E. coli, yeast, insect cells or animal cells can be used as a host cell. When the selection of a host cell is made to prepare a glycoprotein, glycosylation capacity or glycosylation system must be considered first because every host cell has a different glycosylation capacity or glycosylation system. Especially to prepare a human FSH, it is required to select a host cell with an excellent glycosylation capacity because glucose plays an important role in in vivo activity of hormone. For example, among the above-mentioned candidates for a host cell, E. coli has no glycosylation capacity at all. Although yeast or insect cells have a little glycosylation capacity, it is too low to produce a complex type glycoprotein such as human FSH, or it can only produce high mannose type glycoprotein. Therefore, they are not promising candidates for a host cell. Even though mammalian cells have a very good glycosylation capacity or glycosylation system, the level or the type of glycosylation varies from the characteristics of each cell line. So, in order to produce a glycoprotein such as hFSH available for the treatment of a disease, such a mammalian cell line that does not cause antigenicity but is safe and has high in vivo activity must be selected as a host cell.

It is also essential to develop an efficient expression vector for the mass-production of a heterodimeric glycoprotein such as FSH, LH, hCG, TSH, factor VIII and IL-12 by using animal cells. These are the examples of a constituent of such expression vector; promoter playing an important role in controlling the gene expression, intron enabling the increase of transcription efficiency of a gene by stabilizing mRNA, polyadenylation motif (pA) enabling the increase of the stability of mRNA, and selection marker, etc.

Thus, the present inventors prepared an expression vector highly and stably expressing human FSH, and then completed this invention by confirming that FSH could be mass-produced stably by transfecting a host cell having an excellent glycosylation capacity with the prepared expression vector.

### Disclosure

### Technical Problem

It is an object of the present invention to provide an expression vector containing human follicle stimulating hormone gene, a transformant transfected with the expression vector and a method for mass-production of human follicle stimulating hormone by using the same.

### Technical Solution

In order to achieve the above object, the present invention provides a recombinant expression vector containing human FSH gene.

The present invention also provides a transformant transfected with the expression vector.

The present invention further provides a method for mass-production of human follicle stimulating hormone by using the transformant.

Hereinafter, the present invention is described in detail.

The present invention provides a recombinant expression vector containing human FSH gene.

In order to mass-produce heterodimeric glycoprotein such as FSH, LH, hCG, TSH, factor VIII and IL-12 using animal cells, it is essential to develop an efficient expression vector. Thus, the present invention provides a highly efficient expression vector for mass-production of human FSH, considering all the factors involved.

1) A gene coding hFSH is composed of two subunits (alpha and beta subunits) which are transcribed from different genes. In the preferred embodiment of the present invention, IRES (internal ribosomal entry site) of EMCV (encephalomyocarditis virus) was used in order to prepare a heterodimeric glycoprotein by expressing two different glycoproteins in the same cell. Then the resultant was inserted in between alpha subunit gene and beta subunit gene of hFSH to make those genes be expressed simultaneously. So, it is preferred for a gene coding hFSH included in the expression vector of the present invention to contain FSH alpha subunit gene represented by SEQ. ID. No 1 and FSH beta subunit gene represented by SEQ. ID. No 2, and it is more preferred for the gene coding hFSH to have IRES sequence, represented by SEQ. ID. No 7, inserted in between FSH alpha subunit gene and FSH beta subunit gene in order to induce simultaneous expression of both subunit genes.

2) A promoter plays an important role in the regulation of gene expression. So, an expression vector of the present invention was prepared to contain a promoter gene. A promoter included in the expression vector of the present invention is preferably the promoter of early gene of cytomegalovirus (CMV) represented by SEQ. ID. No 8 which has been known as the most powerful promoter.

3) Mammalian genes contain intron. The expression level is remarkably increased in the presence of intron (Korb et al., Nucleic Acids Res, 25:5901-5908, 1993). This might be because that intron can stabilize mRNA and increase transcription efficiency. Thus, an expression vector of the present invention was designed to have intron sequence, and the intron sequence, at that time, was preferably tripartite leader sequence (referred as “TPL” hereinafter) of adenovirus represented by SEQ. ID. No 9.

4) In order to increase the stability of mRNA, polyadenylation motif (pA) is important (Drummond et al., Nucleic Acids Res, 25:7375-7394, 1985). The expression vector of the present invention includes such polyadenylation motif, and the polyadenylation motif is preferred to be polyadenylation motif of early gene of SV40 virus, represented by SEQ. ID. No 13, and/or polyadenylation motif of bovine growth hormone (BGH) gene, represented by SEQ. ID. No 14. In the preferred embodiment of the present invention, an expression vector was prepared by locating BGH pA behind FSH alpha gene, SV40 pA behind Neo gene, and SV40 pA gene behind dihydrofolate reductase (referred as “DHFR”) gene (see FIG. 1).

5) A selection marker gene is necessary to select cell lines that can over-express FSH after being transfected with the expression vector of the present invention. Thus, the expression vector of the present invention was designed to include a selection marker gene, and for the selection marker, DHRF gene, a selection marker represented by SEQ. ID. No 12, was preferably used to facilitate selection of a cell line from CHO/dhrf⁻ cells which were Chinese hamster ovary (CHO) originated cell line. CHO/dhfr⁻ cell line has damaged DHFR gene, indicating that its' nucleic acid synthesizing process is incomplete. But, when the cell is transfected with FSH expression vector of the present invention containing DHFR gene, nucleic acid synthesis can be completed. Thus, it is possible to select those cells expressing FSH by the introduction of FSH expression vector.

Taking all the above-mentioned factors into consideration, the present inventors prepared a recombinant expression vector to express human FSH that includes 1) a gene coding human FSH, 2) a promoter sequence, 3) an intron sequence, 4) a polyadenylation motif sequence, and 5) a selection marker gene.

In the preferred embodiment of the present invention, in order to express both FSH alpha and beta subunits simultaneously in the same cell, IRES was inserted in between FSH alpha subunit gene and FSH beta subunit gene. And the expression efficiency of hFSH was maximized by using a promoter of early gene of cytomegalovirus that has been known as the most powerful promoter. Also, adenovirus tripartite leader sequence (TPL) containing intron to increase the stability and transcription efficiency and polyadenylation motif of early gene of SV40 virus as well as polyadenylation motif of bovine growth hormone gene were all included in the vector of the present invention. In addition, DHFR gene was included as a selection marker to facilitate the selection of cell lines over-expressing FSH from the cells transfected with the expression vector of the present invention.

The present inventors prepared an expression vector containing all the above-mentioned elements and named it ‘Rc/CMV-dhfr-TPL-hFSH beta/alpha’.

The expression vector of the present invention was prepared for the high-expression of human FSH gene, a heterodimeric glycoprotein. And the vector of the invention is very efficient one enabling mass-production of a heterodimeric glycoprotein. It is well known to the people in this field that instead of human FSH gene, any other heterodimeric glycoprotein such as LH, hCG, TSH, factor VIII and IL-12 gene can be included in the expression vector and the resultant vector can be effectively used for the mass-production of a protein coded from the above genes.

The present invention also provides a transformant transfected with the expression vector.

In order to prepare a cell line high-expressing human FSH, which is highly activated in a human body, the present inventors selected Chinese hamster ovary (referred as “CHO” hereinafter) cells among many mammalian cell lines as a host cell line because they have a satisfactory glycosylation capacity or glycosylation system, do not induce antigenicity in vivo, are safe and have a carbohydrate structure showing a high in vivo activity.

Nucleic acid biosynthesizing processes occurring in CHO/dhfr⁻ cells, originated from CHO, are incomplete because DHFR gene therein is damaged. But, nucleic acid biosynthesis is fully recovered in CHO cells when they are transfected with the FSH expression vector of the present invention (Rc/CMV-dhfr-TPL-hFSH beta/alpha) that contains DHFR gene. Therefore, it is possible to select those cells harboring the expression vector by culturing CHO cells in a cell culture medium devoid of nucleic acid. And the cells are growing in a culture medium supplemented with methotrexate (MTX), a substrate analogue of DHFR, by the amplification of DHFR gene in cells. At this time, the amplification of FSH gene is also performed because FSH expression vector of the present invention contains DHFR gene. Thus, adding MTX, a substrate analogue of DHFR, into a cell culture medium can improve the expression level of FSH protein in FSH expressing cells.

In the present invention, CHO/dhfr⁻ cells were transfected with FSH expressing vector, and clones which were highly expressed in high-concentration of MTX and showed homologous were selected and further cultured, resulting in a cell line in which FSH was stably and highly expressed (see FIG. 4 to FIG. 6). The selected cell line high-expressing FSH was named “DPFC325” which was deposited at Korean Cell Line Research Foundation (KCLRF) on Jun. 20, 2003 (Accession No: KCLRF-BP-00082).

The present invention also provides a method for mass-production of human follicle stimulating hormone by using the transformant.

The method for mass-production of human FSH of the present invention includes the following steps:

1) Preparation of an expression vector containing human FSH gene;

2) Transfection of host cells with the expression vector of the step 1);

3) Selection of transformants transfected in the step 2);

4) Selection of a recombinant transformant stably producing FSH from the recombinant transformants selected in the step 3); and

5) Obtainment of FSH from the culture of the recombinant transformant selected in the step 4).

In step 1), the expression vector preferably includes human FSH coding gene, a promoter sequence, an intron sequence, a polyadenylation motif sequence and a selection marker gene, and is more preferably “Rc/CMV-dhfr-TPL-hFSH beta/alpha” constructed in the present invention.

In step 2), the host cell is preferably CHO/dhfr⁻ cell, a CHO originated cell line having incomplete nucleic acid biosynthesizing processes because of the damaged DHFR gene.

In step 4), the recombinant transformant stably producing FSH is preferably “DPFC325” which was deposited at Korean Cell Line Research Foundation (KCLRF) on Jun. 20, 2003 (Accession No: KCLRF-BP-00082).

In step 5), the obtainment of produced FSH is performed by conventional protein purification methods.

### DESCRIPTION OF DRAWINGS

FIG. 1 is a schematic diagram showing the preparation process of Rc/CMV-DHFR-TPL-hFSH beta/alpha vector of the present invention,

FIG. 2 shows cDNA nucleotide sequence and amino acid sequence of hFSH-alpha subunit expressed by pSK-FSH-alpha vector of the present invention,

FIG. 3 shows cDNA nucleotide sequence and amino acid sequence of hFSH-beta subunit expressed by pSK-FSH-beta vector of the present invention,

FIG. 4 is a graph showing the expression of human FSH after transfecting COS-7 cells with Rc/CMV-dhfr-TPL-hFSH beta/alpha vector, confirmed by enzyme-linked immunosorbent assay (ELISA),

FIG. 5 is a graph showing the result of enzyme-linked immunosorbent assay (ELISA), confirming the expression level of human FSH in clones that were selected from cells adapted to 1 uM of MTX by limiting dilution,

FIG. 6 is a photograph showing the result of FISH, confirming whether or not human FSH gene is inserted in chromosome of a CHO cell.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Construction of a Recombinant hFSH Expression Vector (Rc/CMV-dhfr-TPL-hFSH beta/alpha)

<1-1> Preparation of Human FSH Alpha Subunit and Beta Subunit

In order to prepare a structural gene that is essential for the production of a cell line expressing a recombinant human FSH, human FSH alpha subunit and beta subunit genes obtained from human pituitary gland cDNA library were amplified by PCR. More precisely, human pituitary gland cDNA library (cal#HL1139a, Clontech, USA) was used as a substrate, and primers were prepared for PCR with nucleotide sequence of FSH alpha subunit gene represented by SEQ. ID. No 1 and nucleotide sequence of FSH beta subunit gene represented by SEQ. ID. No 2. The primer sequences for PCR amplification of FSH alpha and beta subunit genes were as follows.

1) FSH alpha subunit
Sense primer (SEQ. ID. No 3):
AGCGCCATGGATTACTACAGAAAATAT
(underlined region: Nco I recognition site),
Antisense primer (SEQ. ID. No 4):
GGGGGATCCGGGCCCTTAAGATTTGTGATAATTAACA
(underlined region: BamH I and Apa I recognition
site)
2) FSH beta subunit
Sense primer (SEQ. ID. No 5):
GGGGGCGGCCGCAGGATGAAGACACTCCAGTTT
(underlined region: Not I recognition site),
Antisense primer (SEQ. ID. No 6):
GGGGATATCTTATTCTTTCATTTCACC
(underlined region: EcoR V recognition site)

The whole fraction of pituitary gland cDNA library was used as a substrate, and each pair of primers (alpha subunit: SEQ. ID. No 3 and No 4, beta subunit: SEQ. ID. No 5 and No 6) were used for PCR. PCR was performed as follows; predenaturation at 94° C. for 30 seconds, denaturation at 94° C. for 30 seconds, annealing at 57° C. for 30 seconds, polymerization at 72° C. for 30 seconds, 30 cycles from denaturation to polymerization, and final extension at 72° C. for 5 minutes. The same PCR condition as described above was applied to the amplification of both FSH alpha subunit and FSH beta subunit.

<1-2> Construction of a Vector for Simultaneous Expression of FSH Alpha and Beta Subunit Genes

FSH is composed of two subunits, which are FSH alpha subunit and FSH beta subunit. So, in order to construct an expression vector enabling simultaneous expression of both FSH alpha subunit and FSH beta subunit, IRES (internal ribosomal entry site) of EMCV (encephalomyocarditis virus), represented by SEQ. ID. No 7 was introduced, in which beta subunit was located at the upstream and alpha subunit was located at the downstream.

First, FSH beta subunit gene (390 nucleotides) which was amplified in the above example <1-1> was treated with T4 polynucleotide kinase, then cloned into pSK vector (Stratagene, USA), pretreated with Hinc II, by blunt ligation. The resultant vector was named “pSK-FSH-beta”. Next, FSH alpha subunit gene (351 nucleotides), which had been amplified in the above example <1-1>, was digested with Nco I and BamH I, then inserted in the location of Nco I and BamH I of pSK-IRES vector (Ha et al., Nat. Biotechnol., 20(4):381-386, 2002) containing EMCV IRES, resulting in “pSK-IRES-FSH-alhpa”. FSH-beta DNA fragment, prepared by digesting pSK-FSH beta vector with Xho I and EcoR V, was inserted in pSK-IRES-FSH-alpha vector, which had been digested with Xho I and EcoR V, resulting in the construction of a target vector containing both FSH-alpha subunit gene and FSH-beta subunit gene, which was named “pSK-FSH-alpha/IRES/FSH-beta”.

Lastly, a vector was constructed to express FSH-alpha subunit gene and FSH-beta subunit gene simultaneously in animal cells. For the construction, Rc/CMV plasmid (Invitrogen, USA) harboring a very powerful cytomegalovirus (CMV) promoter, represented by SEQ. ID. No 8, was used as a basic vector. In order to increase the level of expression, adenovirus tripartite leader sequence (TPL), represented by SEQ. ID. No 9, was inserted in pTV2 vector (Lee et al., J. Virol., 72(10):8430-8436, 1998). More precisely, pTV2 vector was used as a template and TPL gene was amplified by PCR using the below primers under the same PCR condition as described in the example <1-1>.

The primer sequences for the amplification of TPL gene are as follows.

Sense primer (SEQ. ID. No 10):
GATATCGATACTCTCTTCC,
Antisense primer (SEQ. ID. No 11):
GCGTCGACCTGCAGTTGGACCTGGGAG
(underlined region: Sal I recognition site)

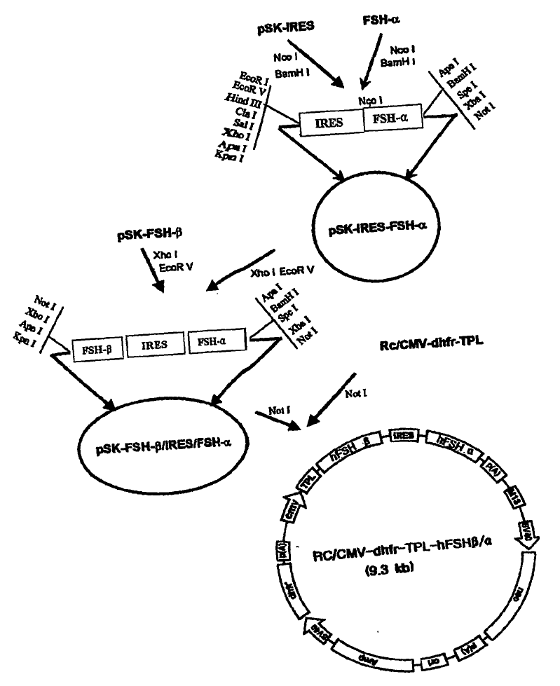

The amplified TPL gene was treated with T4 polynucleotide kinase, which was then inserted in pSK vector (Stratagene, USA) predigested with EcoR V. The resultant product was digested with Sal I, so that the location of Sal I in pSK vector which was corresponding to the area of 5′ of TPL gene and the location of Sal I in 3′ of TPL gene were restricted, resulting in the separation of TPL gene. The separated gene was treated with Klenow enzyme to make it have blunt ends. After digesting with Hind III, Rc/CMV vector (Invitrogen, USA) was also treated with Klenow enzyme to make it have blunt ends, too. The treated vector was linked to the separated TPL gene by the action of ligase, resulting in RcCMV-TPL vector. For the amplification of the gene expression, pSV2-dhfr vector (ATCC 37146) containing mouse dihydrofolate reductase (DHFR) gene, represented by SEQ. ID. No 12, and SV40 pA gene, represented by SEQ. ID. No 13, in the rear of the above gene was digested with Pvu II, then BamH I linker (SEQ. ID. No 15) was inserted in the position. The resultant product was digested with BamH I, resulting in 1.8 kb sized gene containing DHFR gene and SV40 pA gene. The gene was inserted in RcCMV-TPL vector which had been digested with Bgl II, and the final vector was named “Rc/CMV-dhfr-TPL”. PSK-FSH-alpha/IRES/FSH-beta vector was digested with Not I to prepare FSH-alpha/IRES/FSH-beta fragment. The fragment was inserted in the location of Not I of Rc/CMV-dhfr-TPL vector to prepare an expression vector for high-expression of FSH. The product was named “Rc/CMV-dhfr-TPL-hFSH beta/alpha” (FIG. 1). DNA sequencing of FSH-alpha subunit gene and FSH-beta subunit gene in Rc/CMV-dhfr-TPL-hFSH beta/alpha plasmid was performed by the method of Sanger et al. (Sanger et al., Proc. Natl. Acad. Sci. USA, 74:5463-5467, 1977).

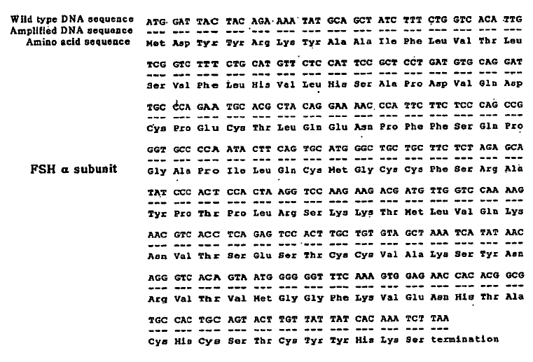

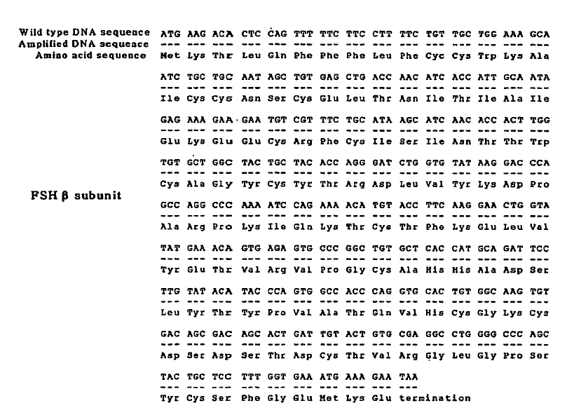

As a result, DNA sequences of FSH-alpha subunit and FSH-beta subunit that were inserted in the expression vector of the present invention were confirmed to be identical with the reported DNA sequences of FSH-alpha subunit and FSH-beta subunit, indicating that the expression vector of the present invention was correctly constructed (FIG. 2 and FIG. 3).

### EXAMPLE 2

### Preparation of a Cell Line Expressing Human FSH

A cell line was transfected with the recombinant human FSH expression vector (Rc/CMV-dhfr-TPL-hFSH beta/alpha), constructed in the above example 1, to investigate the expression of recombinant human FSH. Particularly, COS-7 cells (ATCC, catalog # CRL-1651), being under culture, were inoculated into 60 mm dish plates (5×10⁵ cells/plate), followed by further culture for 18 hours. The culture medium (DMEM+10% FBS) was replaced with a fresh medium (4.5 ml). Then, about 4 hours later, the cells were transfected with a FSH expression vector Rc/CMV-dhfr-TPL-hFSH beta/alpha plasmid or a control vector Rc/CMV-dhfr-TPL plasmid by means of CaPO₄ coprecipitation. More precisely, 10 μg of each DNA was mixed with pure distilled water, making the final volume 225 μl. Then, 25 μl of 2.5 M CaCl₂ solution was added and mixed by vortexer. 250 μl of 2×HBS solution (280 mM NaCl, 10 mM KCl, 1.5 mM Na₂HPO₄2H₂O, 12 mM dextrose, 50 nM HEPES) was slowly dropped drop by drop into a tube containing the DNA. Reaction was induced for 10 minutes at room temperature. The mixture was transferred onto a cell culture dish, followed by further culture for 6 hours. The dish was washed with PBS (phosphate buffered saline) three times and then a fresh medium was supplemented, followed by further culture for 48 more hours.

### EXAMPLE 3

### Confirmation of hFSH Expression by ELISA

Temporarily over-expressed FSH was quantified by using the culture medium of COS-7 cells transfected with Rc/CMV-dhfr-TPL-hFSH beta/alpha plasmid or Rc/CMV-dhfr-TPL (control plasmid) in the above example 2. ELISA (enzyme-linked immunosorbent assay) kit (MEDI-CORP, cat# KTSF 3651, Canada) was used to detect FSH, by following the manufacturer's protocol. Particularly, 100 μl of the transfected cell culture medium cultured for 48 hours and 100 μl of standard solution equipped in the kit were added to the well coated with anti-human FSH, leading to the reaction for 30 minutes on a plate shaker. The wells were washed with washing solution provided by the manufacturer, and then, anti-human FSH antibody linked to HRP (horse radish peroxidase) was added to each well by 100 μl, followed by reaction on the plate shaker at room temperature. After 30 minutes of reaction, the plate was washed with washing solution three times. Then, 100 μl of substrate solution was added to each well and reaction was induced at room temperature on the plate shaker. 15 minutes later, 100 μl of stop solution was added to each well and OD₄₅₀ was measured. Using the standard solution provided by the manufacturer of the kit, standard curve and function were obtained. The amount of FSH was quantified by using the standard curve and the function.

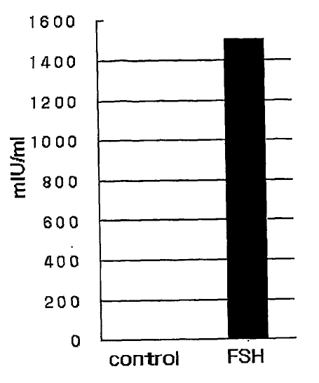

As a result, human FSH was hardly shown in cells transfected with Rc/CMV-dhfr-TPL, a control plasmid, but human FSH was over-expressed in cells transfected with Rc/CMV-dhfr-TPL-hFSH beta/alpha plasmid (FIG. 4).

### EXAMPLE 4

### Preparation of a CHO Cell Line Expressing hFSH

Rc/CMV-dhfr-TPL-hFSH beta/alpha vector, which was proved in the above example 3 to over-express hFSH, was introduced into Chinese hamster ovary cells (CHO/dhfr⁻) (Urlaub et al., Somat. Cell Mol. Genet., 12:555-566, 1986) having incomplete nucleic acid biosynthesizing process resulted from the damaged DHFR gene, leading to the preparation of a cell line suitable for mass-production of hFSH protein. More precisely, before a hFSH over-expressing vector was introduced, CHO/dhfr⁻ cells were thawed and put in a T-75 culture flask, followed by culture in MEM-alpha medium (GIBCO BRL, USA) supplemented with HT supplement (GIBCO BRL, USA) and 10% dialyzed fetal bovine serum (FBS) until cell density reached 80-90%. Trypsin was treated to those cells growing with being adhered to the plate. Then, cells were recovered and 5×10⁵ cells were plated on 60 mm dish. The cells were transfected with Rc/CMV-dhfr-TPL-hFSH beta/alpha, a FSH expression vector, by means of CaPO₄ coprecipitation explained in the above example 3, followed by further culture for two more days. After the completion of the culture, the transfected CHO/dhfr⁻ cells were diluted consecutively (1/2, 1/5, 1/10, 1/20, 1/50, 1/100, 1/200, 1/500), and then, cultured in HT supplement-free medium on 100 mm dish, during which the primary screening was performed. During screening, the medium was replaced once a week, passage was omitted, and plates with colony formed were selected after 2 to 3 weeks. Colonies were separated from the plates and transferred into a 24-well plate for subculture. About one or two weeks later, cell clones in the 24-well plate were treated with trypsin. Then, cells were recovered and plated again in a 24-well plate (2×10⁴ cells/well). 48 hours later, supernatant was obtained to measure the expression level of FSH by using FSH detecting ELISA kit (MEDI-CORP, cat# KTSF 3651, Canada). Cell clones showing higher level of FSH expression were diluted consecutively, which were put in a 100 mm dish for further culture. 20 nM of methotrexate (MTX) was added to the medium for the second screening. Finally, clones high-expressing human FSH were selected with the same identifying processes as used for the primary screening.

### EXAMPLE 5

### Selection of a CHO Cell Line Expressing hFSH High

CHO/dhfr⁻ cells deficient in DHFR cannot survive in MEM-alpha medium, a minimum essential medium devoid of nucleic acid, because the cells have an incomplete nucleic acid biosynthesizing process. However, recombinant CHO cells in which FSH gene and DHFR gene are inserted, recover nucleic acid biosynthesizing capability, so that the MEM-alpha medium can be used for the selection culture. Thus, in the present invention, in order to select cell lines expressing hFSH high, clones prepared in the above example 4 were cultured in culture media having different MTX concentrations (20 nM to 1 μM). More precisely, the primary selected clones were subcultured in MEM-alpha medium supplemented with 10% of dialyzed FBS, during which the concentration of MTX was increased gradually. Clones were cultured at different MTX concentrations to select specific ones having resistance against MTX. The supernatants of each clone in different culture stages were obtained to measure the expression of FSH, resulting in the second selection of cells expressing FSH high. The expression of FSH was quantified by means of ELISA described in the above example 3.

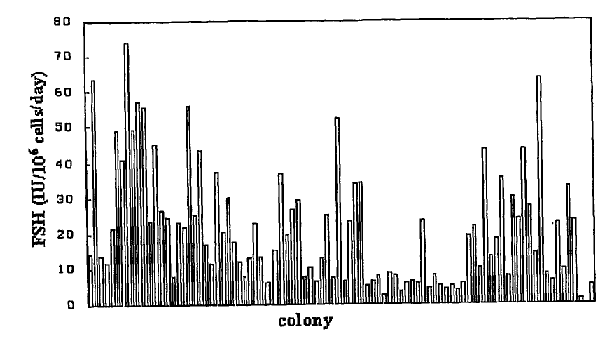

For the third selection, monoclonal selection was performed by means of limiting dilution method to reduce the chance of heterogeneity during MTX amplification. That is, single cells having even FSH expression rate and excellent productivity were separated from all the clones showing irregular FSH expression rates by means of limiting dilution method. Cells were distributed in a 96-well plate by one-two clones per each well, and then the wells inoculated with 1 cell only were selected, followed by further culture for 2 to 3 weeks. From the plate showing colonies formed, cells were separated and sub-cultured to measure the amount of expressed FSH by ELISA. At last, single cells highly expressing FSH were separated (FIG. 5). Among those separated cells through the third selection, a recombinant CHO cell line showing a stable proliferation in the presence of 1 μM MTX and high FSH productivity was selected and named “DPFC325”, which was deposited at Korean Cell Line Research Foundation (KCLRF), Seoul, Korea on Jun. 20, 2003 (Accession No: KCLRF-BP-00082 [symbol] ).

### EXAMPLE 6

### Confirmation of the Insertion of hFSH Gene in Chromosome by FISH

For the clones used for the production of a protein as a treating agent, genetic stability is so important. In order for a plasmid vector inserted in a cell line to be transmitted to the next generation stably, integration in chromosome of CHO cell is essential. In the present invention, FISH (fluorescent in situ hybridization) was performed to investigate whether FSH gene was inserted in chromosome of a recombinant cell line or not. Precisely, metaphase chromosome of CHO cell and nucleus of interphase were hybridized with biotin-labeled probe. The hybridized metaphase chromosome was reacted with avidin conjugated with a fluorescent material, followed by observation.

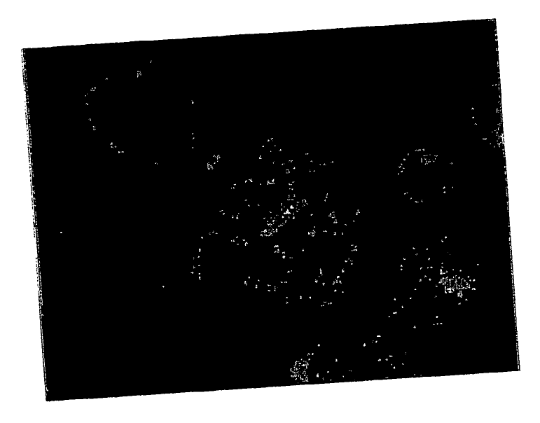

As a result, almost all nuclei of interphase showed reaction color, indicating that FSH gene was successfully inserted in chromosome (FIG. 6).

### INDUSTRIAL APPLICABILITY

As explained hereinbefore, a transformant of the present invention can be effectively used for the treatment of infertility since the transformant can produce human follicle stimulating hormone stably and largely.

### Sequence List Text

Nucleotide sequences represented by SEQ. ID. No 1 to SEQ. ID. No 15 are the ones used for the construction of recombinant hFSH expression vector (Rc/CMV-dhfr-TPL-hFSH beta/alpha) of the example 1.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. An expression vector to express human follicle stimulating hormone (FSH) comprising a gene coding human FSH, a promoter sequence, a polyadenylation motif sequence and a dihydrofolate reductase (DHFR) gene.

2. The expression vector as set forth in claim 1, wherein the human FSH gene consists of human FSH alpha subunit gene represented by SEQ. ID. No 1, internal ribosomal entry site (IRES) sequence represented by SEQ. ID. No 7 and human FSH beta subunit gene represented by SEQ. ID. No 2.

3. The expression vector as set forth in claim 1, wherein the promoter is the promoter of early gene of cytomegalovirus (CMV) represented by SEQ. ID. No 8.

4. The expression vector as set forth in claim 1, wherein tripartite leader sequence of adenovirus represented by SEQ. ID. No 9 is additionally included.

5. The expression vector as set forth in claim 1, wherein the polyadenylation motif is a polyadenylation motif of early gene of SV40 virus, represented by SEQ. ID. No 13, and/or a polyadenylation motif of bovine growth hormone (BGH) gene, represented by SEQ. ID. No 14.

6. The expression vector as set forth in claim 1, wherein the dihydrofolate reductase (DHFR) gene is a base sequence represented by SEQ. ID. No 12.

7. The expression vector as set forth in claim 1, wherein the vector is RC/CMV-dhfr-TPL-hFSH beta/alpha represented in FIG. 1.

8. A recombinant transformant mass-producing human FSH prepared by introducing the expression vector of claim 1 into host cells.

9. The recombinant transformant as set forth in claim 8, wherein the host cell is a CHO (Chinese hamster ovary) originated cell line (CHO/dhfr⁻) harboring damaged dihydrofolate reductase (DHFR) gene.

10. A recombinant transformant DPFC325 (Accession No: KCLRF-BP-00082) mass-producing human FSH prepared by introducing the expression vector of claim 7 into a CHO originated cell line (CHO/dhfr⁻).

11. A method for mass-production of human follicle stimulating hormone comprising the following steps of:
1) preparing an expression vector containing human FSH gene;
2) transfecting host cells with the expression vector of step 1);
3) selection of selecting transformants transfected in step 2);
4) selecting a recombinant transformant stably producing human FSH from the recombinant transformants selected in the step 3); and
5) obtaining human FSH from the culture of the recombinant transformant selected in step 4).

12. The method for mass-production of human follicle stimulating hormone as set forth in claim 11, wherein the expression vector of step 1) is an expression vector to express human follicle stimulating hormone (FSH) comprising a gene coding human FSH, a promoter sequence, a polyadenylation motif sequence and a dihydrofolate reductase (DHFR) gene.

13. The method for mass-production of human follicle stimulating hormone as set forth in claim 11, wherein the host cell of step 2) is a CHO originated cell line (CHO/dhfr⁻) harboring damaged dihydrofolate reductase (DHFR) gene.

14. The method for mass-production of human follicle stimulating hormone as set forth in claim 11, wherein the selected recombinant transformant of step 4) is a transformant mass-producing human FSH prepared by introducing the expression vector to express human follicle stimulating hormone (FSH) comprising a gene coding human FSH, a promoter sequence a polyadenylation motif sequence and a dihydrofolate reductase (DHFR) gene into host cells.

15. The method for mass-production of human follicle stimulating hormone as set forth in claim 11, wherein the selected recombinant transformant of step 4) is the transformant mass-producing human FSH prepared by introducing the expression vector to express human follicle stimulating hormone (FSH) comprising a gene coding human FSH, a promoter sequence, a polyadenylation motif sequence and a dihydrofolate reductase (DHFR) gene into host cells, wherein the host cell is a CHO (Chinese hamster ovary) originated cell line (CHO/dhfr⁻) harboring damaged dihydrofolate reductase (DHFR) gene.

16. The method for mass-production of human follicle stimulating hormone as set forth in claim 11, wherein the selected recombinant transformant of step 4) is a recombinant transformant DPFC325 (Accession No: KCLRF-BP-00082) mass-producing human FSH prepared by introducing the expression vector RC/CMV-dhfr-TPL-hFSH beta/alpha represented in FIG. 1 into a CHO originated cell line (CHO/dhfr⁻).
